(19) United States
(12) Patent Application Publication
(10) Pub. No.: US 2006/0025865 A1
(43) Pub. Date: Feb. 2, 2006

(54) Implant

(75) Inventors: Jan Reich, Hochemmingen (DE); Thomas Hagen, Tuttlingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/196,807

(22) Filed: Aug. 2, 2005

Related U.S. Application Data
(63) Continuation of application No. PCT/EP04/00274, filed on Jan. 16, 2004.

(30) Foreign Application Priority Data
Feb. 5, 2003 (DE) 103 05 591.6

(51) Int. Cl. A61F 2/38; A61F 2/30

(52) U.S. Cl. 623/20.35; 623/23.42; 623/20.3

(57) ABSTRACT

In order to improve an implant for the replacement of at least a part of a natural articular surface of a bone forming part of a joint comprising an anchorage surface abutting against the remaining bone and an artificial articular surface directed away from the bone in such a way that as little bone substance as possible should be removed in a simple manner for insertion of the implant, it is proposed that the anchorage surface comprises at least one cylindrically shaped area, which defines a cylinder axis.

This application is a continuation of international application number PCT/EP2004/000274 filed on Jan. 16, 2004.

The present disclosure relates to the subject matter disclosed in international application number PCT/EP2004/000274 of Jan. 16, 2004 and German application number 103 05 591.6 of Feb. 5, 2003, which are incorporated herein by reference in their entirety and for all purposes.

### BACKGROUND OF THE INVENTION

The invention relates to an implant for the replacement of at least a part of a natural articular surface of a bone forming a part of a joint comprising an anchorage surface abutting against the remaining bone and an artificial articular surface directed away from the bone.

Such implants are used, for example, with artificial knee joints. These implants enable replacement of the articular surfaces of the femur, i.e. one or both condyles of the femur, damaged as a result of excessive attrition.

Anchorage surfaces of such known implants are spherically shaped or formed from several plane surfaces inclined relatively to one another, as described in European Patent EP 0 327 387 B1.

A disadvantage with implants with several plane articular surfaces has proved to be that more bone substance than actually necessary has to be removed prior to insertion of the implant. While this is not the case with implants with spherically shaped anchorage surfaces, a spherical cutter head must be inserted from ventral direction to cut the bone. This makes it necessary to open the knee joint almost completely to prepare the bone of the femur for insertion of the implant.

Therefore, it is an object of the present invention to improve an implant of the aforementioned type such that as little bone substance as possible should be removed in a simple manner.

### SUMMARY OF THE INVENTION

This object is achieved according to the invention with an implant of the aforementioned type in that the anchorage surface comprises at least one cylindrically shaped area, which defines a cylinder axis.

Because the implant comprises a cylindrically shaped area, only a cylindrical surface corresponding to the anchorage surface has to be prepared on the bone, in which case less bone substance has to be removed than would have been necessary for the insertion of implants with several plane anchorage surfaces. Moreover, a cylindrical surface has the advantage that this can be cut from lateral or medial direction, and not only from ventral direction. This means it is no longer absolutely necessary to open the entire knee, instead a minimally invasive procedure on the knee joint is possible in this way.

It is beneficial if the entire anchorage surface is cylindrically shaped. The structure of the implant, and thus also the production thereof, is simplified as a result of this. Moreover, the least amount of bone substance needs to be resected for insertion of such an implant.

It is advantageous if the articular surface is convexly curved directed away from the cylinder axis. This configuration enables the articular surface to be configured very closely to the natural shape.

The articular surface preferably has a spherical shape. A spherical shape is particularly simple to produce and can be simply guided by a spherically recessed slide element serving as an artificial meniscus essentially completely during a flexing movement of the knee.

A particularly simple structure of the implant results if the cylinder axis contains the centre point of the spherically shaped articular surface. Moreover, a cup-shaped configuration of the implant thus results, which renders minimal resection of bone substance necessary.

According to a preferred embodiment of the invention, it can be provided that the anchorage surface comprises a plane area, and that the plane area adjoins the cylindrically shaped area. Undercuts on the bone to be resected can be prevented as a result of the provision of the plane area of the anchorage surface.

It is beneficial if in the transition area between the plane area and the cylindrically shaped area, the plane area forms a tangential plane onto the cylindrically shaped area. As a result of this, the anchorage surface can be configured to be completely crack- and edge-free. However, if in the transition area between the plane area and the cylindrically shaped area the plane area is moulded to the cylindrically shaped area at an angle to a tangential plane, then the implant adapts to the remaining bone surface particularly well if the plane surface is tilted a few degrees dorsally, e.g. by 10°. In this way, only a minimal amount of bone substance needs to be removed. In addition, undercuts are prevented.

According to a preferred embodiment of the invention, it can be provided that the implant has a plane anchorage section and a curved anchorage section, that the plane anchorage section comprises the plane area and that the curved anchorage section comprises the cylindrically shaped area. This results in a particularly simple structure of the implant.

The structure of the implant is even simpler if the plane anchorage section has a plane of symmetry, which extends transversely to the cylinder axis. The curved anchorage section can be shaped as desired in this case.

The implant is particularly simple to produce and simple in structure if the curved anchorage section has a plane of symmetry, which extends transversely to the cylinder axis.

The implant preferably has a plane of symmetry, which extends transversely to the cylinder axis. This results in a particularly simple structure of the implant overall both with respect to design and to production technique.

It is beneficial if the implant has side faces, which connect the articular surface and the anchorage surface to one another. This enables the formation of edges in the transition area between the articular surface and the anchorage surface to be avoided or stop surfaces to be configured in the shape of the side faces.

A particularly small amount of bone substance needs to be resected if the side faces are at a constant spacing from one another parallel to the direction of the cylinder axis.

According to a further preferred embodiment of the invention it can be provided that the side faces are curved at least in sections, so that a spacing of the side faces from a plane running transversely to the cylinder axis varies. This results in a structure of the implant, which requires a minimal amount of bone substance to be resected, depending on the natural condyle to be replaced. Moreover, condyle surfaces can thus be created in the most natural way in a majority of patients.

A shape of the implant that particularly resembles a natural condyle shape results if starting from the plane anchorage section, the spacing of the side faces increases in a direction towards the plane extending transversely to the cylinder axis.

In principle, the side faces of the curved anchorage section could be oriented parallel in relation to the plane extending transversely to the cylinder axis. It is beneficial if the side faces of the curved anchorage section are curved in relation to the plane extending transversely to the cylinder axis. This allows a natural condyle shape to be substantially reconstructed.

A particularly simple structure of the implant results if a curvature of the side faces is constant.

It is advantageous if the spacing between the articular surface and the cylindrically shaped area of the anchorage surface is constant in a plane extending transversely to the cylinder axis. Only a minimal resection of the bone is required prior to insertion of the implant as a result of this.

It is beneficial if the spacing between the articular surface and the cylindrically shaped area of the anchorage surface is at its greatest in a plane of symmetry of the implant extending transversely to the cylinder axis. The stability of the implant can be advantageously increased in this way. In addition, the amount of bone to be removed is minimised as a result of the symmetrical configuration of the implant.

It is particularly advantageous if the cylindrically shaped area of the anchorage surface extends at maximum over an angle range of 180°. As a result, undercuts of the implant on the bone forming a part of the joint are avoided. The angle range advantageously extends over 120° at maximum.

A particularly good adhesion of the implant on the femur results if the angle range comprises about 110°.

According to a preferred embodiment of the invention it can be provided that the implant is a unicondylar implant for replacement of a condyle surface of a femur. This configuration of the implant allows only a part of the bone of the femur, i.e. only or of the two condyles present, to be replaced in a simple manner if this is sufficient as a result of excessive attrition of the natural articular surface on one side.

To allow always the optimum implant to be inserted irrespective of the size of the joint during an operation, in which a joint has to be at least partially replaced, it is advantageous if a set of implants is provided, wherein radii of curvature of the articular surfaces respectively vary by 5 mm, especially by 3 mm, preferably by 1 mm.

In order to replace both lateral and medial condyle surfaces individually, where required, it is advantageous if the implant set comprises implants configured symmetrically to one another.

The following description of a preferred embodiment of the invention serves the purpose of more detailed explanation in association with the drawing.

### BRIEF DESCRIPTION OF THE DRAWINGS

FIG. 1 is a perspective view of the implant;

FIG. 2 is a further perspective view of the implant from FIG. 1;

FIG. 3 is a perspective view of a partially resected distal end of a femur;

FIG. 4 is a side view of the femur from FIG. 3 with an implant arranged thereon;

FIG. 5 is a plan view onto a plane anchorage surface of a second embodiment of an implant;

FIG. 6 is a view of the implant from FIG. 5 in the direction of arrow A;

FIG. 7 is a view of a femur from the front with an implant shown in FIGS. 5 and 6 arranged thereon, and

FIG. 8 is a view similar to FIG. 7 with a third embodiment of an implant.

### BRIEF DESCRIPTION OF THE DRAWINGS

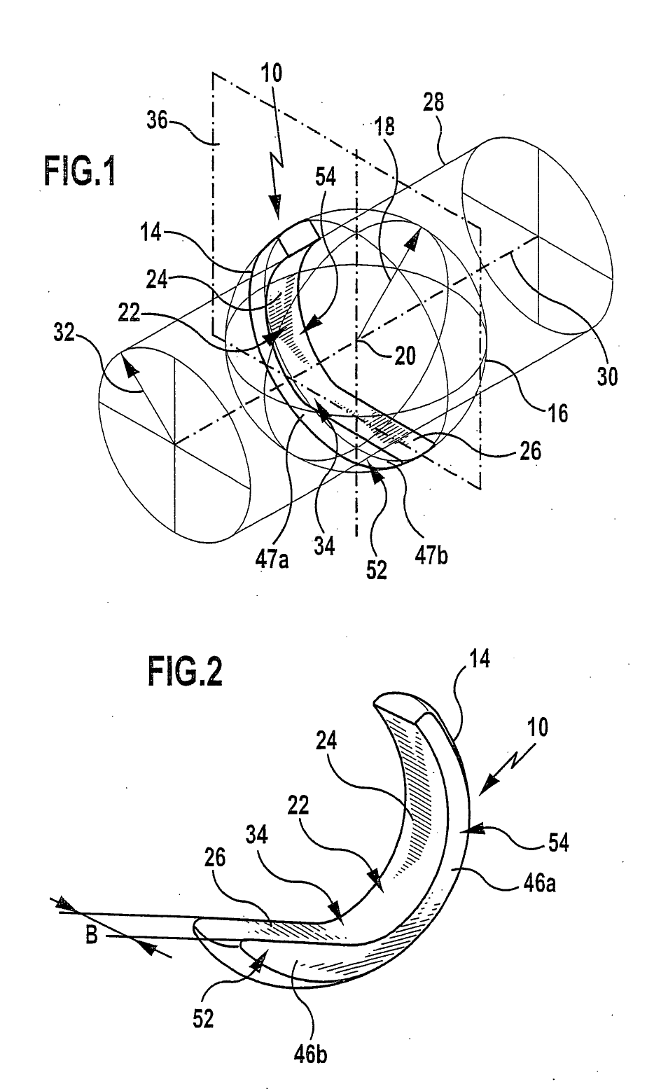

FIGS. 1 and 2 show a unicondylar implant given the overall reference 10 for the replacement of at least a part of a natural condyle surface of a femur 12 forming part of a knee joint. The implant 10 has a convexly curved artificial articular surface 14, which is also referred to as articulation surface. The articular surface 14 forms a section of a sphere 16 with a sphere radius 18 and a sphere centre point 20.

An anchorage surface given the overall reference 22 has a concave area 24 and a plane area 26. The concave area is part of a cylinder 28, the axis of symmetry, i.e. the cylinder axis 30, of which contains the sphere centre point 20. The concave area 24 extends over an angle range 25, which is smaller than 180°, preferably amounts to about 110°, as in the embodiment shown in FIGS. 1, 2 and 4. The cylinder radius 32 is slightly smaller than the sphere radius 18. The concave area 24 merges tangentially into the plane area 26 in a transition area 34, i.e. without formation of an edge.

Moreover, the implant 10 has side faces 46a and 46b as well as 47a and 47b, side faces 46a and 47a connecting the articular surface 14 in the area of a curved anchorage section 54 of the implant 10 to the concave area 24, side faces 46b and 47b connecting the articular surface 14 to the plane area 26 in the area of a plane anchorage section 52 of the implant 10. In this case, the spacing B of side faces 46a and 47a from one another as well as spacing B of side faces 46b and 47b from one another is essentially constant. Moreover, side faces 46 and 47 run essentially parallel to one another.

The implant 10 is configured overall mirror-symmetrically to a mirror plane 36, which is oriented perpendicular to the cylinder axis 30 and contains the sphere centre point 20.

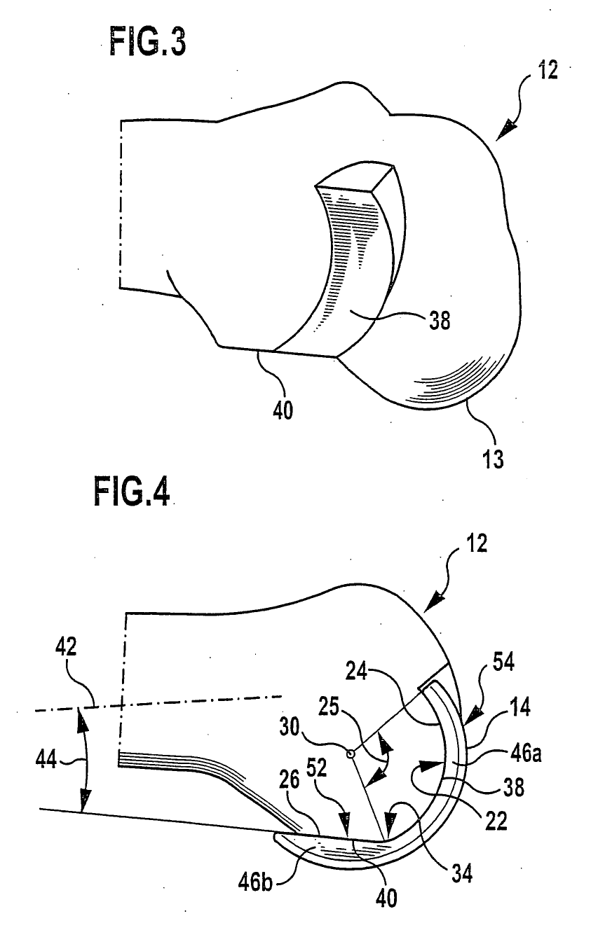

To connect the implant 10 to the bone of the femur 12, one of the two originally present condyles 13 is partially resected, i.e. in such a manner that a convexly shaped, cylindrical abutment surface 38 remains on the bone. Adjoining this abutment surface 38 shown in FIG. 3 is a plane section 40 on the bone, which is configured so that it essentially corresponds to the plane area 26 of the implant 10. The abutment surface 38 and in particular the plane section 40 are arranged such that a plane defined by the plane section 40 is tilted dorsally in relation to a longitudinal axis 42 of the femur by an angle 44, which ideally amounts to about 100. Undercuts on the bone are prevented as a result of this.

The largest spacing between the anchorage surface 22 and the articular surface 14 lies in the plane of symmetry 36, in particular between the plane area 26 and the articular surface 14.

The abutment surface 38 can be prepared with a laterally placed cutting tool, for example. It is not necessary to open the knee joint from ventral direction in this case. Moreover, the implant 10 can also be brought to the abutment surface 38 and the plane section 40 of the femur 12 from the lateral or medial direction and cemented as required.

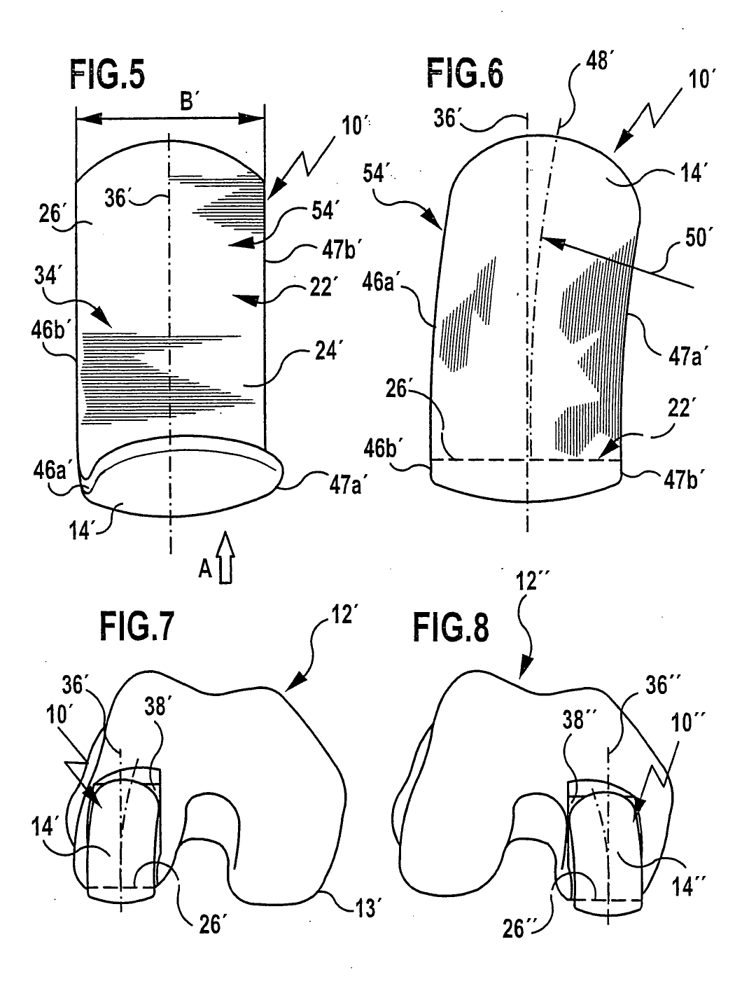

FIGS. 5 to 7 show a second embodiment of an implant according to the invention and given the overall reference 10′. It corresponds essentially to the implant 10 described in association with FIGS. 1 to 4, and therefore the same parts of implants 10 and 10′ are provided with the same reference numerals.

Contrary to implant 10, implant 10′ is not completely symmetrical in relation to a plane of symmetry 36′. Only the part of the implant 10′, which comprises the plane area 26′, i.e. the plane anchorage section 52′, is configured symmetrically to the plane 36′. Conversely, the curved anchorage section 54′ comprising the concave area 24′ is curved in relation to the plane of symmetry 36′, i.e. so that a centre of gravity of the curved anchorage area 54′ lies not on but near the plane of symmetry 36′. Side faces 46a′ and 47a′ or 46b′ and 47b′, which connect the convexly curved artificial articular surface 14′ to the concave area 24′ and the plane area 26′, have a constant spacing B′ from one another, so that implant 10′, like implant 10, has a constant width transversely to the plane of symmetry 36′.

The articular surface 14′ is spherically shaped, so that in the case of the likewise cylindrically shaped concave area 24′ the width of side face 46a′ transversely to articular surface 14′ is larger than the width of side face 47a′. As shown in FIG. 6, a centre line 48′ of the implant 10′ is curved away from the plane of symmetry 36′. The radius of curvature 50′ of the centre line 48′ amounts to about 150 mm and is constant, but can also decrease or increase with increasing distance from the plane area 26′.

As shown in FIG. 7, implant 10′ replaces a partially resected natural condyle 13′ of the femur 12′, in which case because of the curved anchorage area 54′ a better adaptation to the natural conditions is achieved than with implant 10.

Because of the unsymmetrical structure of implant 10′ overall, two differently curved implants are required for an optimum reconstruction of both natural condyle surfaces of a femur. An example of an implant 10″ configured mirror-symmetrically overall to implant 10′ is shown in FIG. 8. This allows a condyle surface to be replaced in keeping with the natural anatomy.

1. Implant for the replacement of at least a part of a natural articular surface of a bone forming a part of a joint comprising an anchorage surface abutting against the remaining bone and an artificial articular surface directed away from the bone, wherein the anchorage surface comprises at least one cylindrically shaped area, which defines a cylinder axis.

2. Implant according to claim 1, wherein the entire anchorage surface is cylindrically shaped.

3. Implant according to claim 1, wherein the articular surface is convexly curved directed away from the cylinder axis.

4. Implant according to claim 3, wherein the articular surface has a spherical shape.

5. Implant according to claim 4, wherein the cylinder axis contains the centre point of the spherically shaped articular surface.

6. Implant according to claim 1, wherein the anchorage surface comprises a plane area, and that the plane area adjoins the cylindrically shaped area.

7. Implant according to claim 6, wherein in the transition area between the plane area and the cylindrically shaped area the plane area forms a tangential plane onto the cylindrically shaped area.

8. Implant according to claim 6, wherein the implant has a plane anchorage section and a curved anchorage section, that the plane anchorage section comprises the plane area, and that the curved anchorage section comprises the cylindrically shaped area.

9. Implant according to claim 8, wherein the plane anchorage section has a plane of symmetry, which extends transversely to the cylinder axis.

10. Implant according to claim 8, wherein the curved anchorage section has a plane of symmetry, which extends transversely to the cylinder axis.

11. Implant according to claim 1, wherein the implant has a plane of symmetry, which extends transversely to the cylinder axis.

12. Implant according to claim 6, wherein the implant has a plane of symmetry, which extends transversely to the cylinder axis.

13. Implant according to claim 1, wherein the implant has side faces, which connect the articular surface and the anchorage surface to one another.

14. Implant according to claim 6, wherein the implant has side faces, which connect the articular surface and the anchorage surface to one another.

15. Implant according to claim 13, wherein the side faces are at a constant spacing from one another parallel to the direction of the cylinder axis.

16. Implant according to claim 13, wherein the side faces are curved at least in sections, so that a spacing of the side faces from a plane extending transversely to the cylinder axis varies.

17. Implant according to claim 16, wherein, starting from the plane anchorage section, the spacing of the side faces increases in a direction towards the plane extending transversely to the cylinder axis.

18. Implant according to claim 16, wherein the side faces of the curved anchorage section are curved in relation to the plane extending transversely to the cylinder axis.

19. Implant according to claim 18, wherein a curvature of the side faces is constant.

20. Implant according to claim 1, wherein the spacing between the articular surface and the cylindrically shaped area of the anchorage surface is constant in a plane extending transversely to the cylinder axis.

21. Implant according to claim 6, wherein the spacing between the articular surface and the cylindrically shaped area of the anchorage surface is constant in a plane extending transversely to the cylinder axis.

22. Implant according to claim 1, wherein the spacing between the articular surface and the cylindrically shaped area of the anchorage surface is at its greatest in a plane of symmetry of the implant extending transversely to the cylinder axis.

23. Implant according to claim 6, wherein the spacing between the articular surface and the cylindrically shaped area of the anchorage surface is at its greatest in a plane of symmetry of the implant extending transversely to the cylinder axis.

24. Implant according to claim 1, wherein the cylindrically shaped area of the anchorage surface extends at maximum over an angle range of 180°, in particular over an angle range of 120° at maximum.

25. Implant according to claim 6, wherein the cylindrically shaped area of the anchorage surface extends at maximum over an angle range of 180°, in particular over an angle range of 120° at maximum.

26. Implant according to claim 25, wherein the angle range comprises about 110°.

27. Implant according to claim 1, wherein the implant is a unicondylar implant for replacement of a condyle surface of a femur.

28. Implant according to claim 6, wherein the implant is a unicondylar implant for replacement of a condyle surface of a femur.

29. Implant comprising a plurality of implants according to claim 1, wherein radii of curvature of the articular surfaces vary by a spacing of 5 mm, especially by 3 mm, preferably by 1 mm.

30. Implant comprising a plurality of implants according to claim 6, wherein radii of curvature of the articular surfaces vary by a spacing of 5 mm, especially by 3 mm, preferably by 1 mm.

31. Implant comprising a plurality of implants according to claim 19, wherein radii of curvature of the articular surfaces vary by a spacing of 5 mm, especially by 3 mm, preferably by 1 mm.

32. Implant comprising a plurality of implants according to claim 20, wherein radii of curvature of the articular surfaces vary by a spacing of 5 mm, especially by 3 mm, preferably by 1 mm.

33. Implant comprising a plurality of implants according to claim 22, wherein radii of curvature of the articular surfaces vary by a spacing of 5 mm, especially by 3 mm, preferably by 1 mm.

34. Implant according to claim 29, wherein the implant set comprises implants configured symmetrically to one another.

35. Implant according to claim 30, wherein the implant set comprises implants configured symmetrically to one another.
